(19) United States
(12) Patent Application Publication
(10) Pub. No.: US 2008/0133148 A1
(43) Pub. Date: Jun. 5, 2008

(54) Method For Checking Indoor Environment

(75) Inventors: Tomio Uchi, Tokyo (JP); Takuo Kobayashi, Kanagawa (JP); Yuichi Imai, Tokyo (JP); Hiroshige Kawakami, Hyogo (JP)

Correspondence Address: GREENBLUM & BERNSTEIN, P.L.C., 1950 ROLAND CLARKE PLACE, RESTON, VA, 20191, US

(73) Assignees: Yukio YANAGISAWA, Tokyo (JP); IMAI CORPORATION, Okayama (JP); NIPPON LIVING COMPANY, LTD., Tokyo (JP)

(21) Appl. No.: 11/718,250

(22) PCT Filed: Oct. 25, 2005
(86) PCT No.: PCT/JP05/19544

§ 371 Date: Jul. 18, 2007

(30) Foreign Application Priority Data
Nov. 1, 2004 (JP) 2004-318010

(51) Int. Cl. G01N 33/00

(52) U.S. Cl. 702/24

(57) ABSTRACT

In assessing the effect of indoor emission sources, such as furniture and building materials which emit hazardous chemical substances to indoor environment, with respect to chemical substances emitted from each emission source; calculating a emission amount (Qn) of each emission source per time using parameter of a emission rate (Fn) of chemical substances emitted from each emission source per area and per time and a surface area (Sn) of each emission source; with the result, calculating individually an indoor concentration of a specific chemical substance when assuming that only each emission source is individually placed indoors; and outputting the individual indoor concentration as the fundamental data.

### TECHNICAL FIELD OF THE INVENTION

The present invention relates to a method for checking an indoor environment to evaluate the effect of each emission source in an indoor environment that is contaminated with hazardous chemical substances such as formaldehyde released from indoor emission sources including furniture and building materials.

### BACKGROUND ART

In recent years, it has been reported that residents living in new housing suffer from a variety of poor physical conditions such as headaches, throat irritation, eye irritation, nasal inflammation, vomiting, breathing problems, dizziness and skin irritation. They are referred to as “sick building syndrome”, and cause a social issue.

While the onset mechanism of the sick building syndrome remains partially unelucidated, it is considered to be mainly attributable to indoor air pollution that is caused by the release of hazardous chemical substances such as formaldehyde or volatile organic compounds (VOC) contained in building materials, furniture, furnishing goods, carpets, curtains, and so on.

Although a resident suffers from such sick building syndrome or indoor concentration of volatile chemicals is measured as high, it is difficult to specify an emission source and we couldn't know how to act to reduce the indoor levels.

In a case of furniture, it is not impossible to specify the emission source by measuring an indoor concentration in a state where an article of furniture is removed, but building materials such as flooring, walling and ceiling materials which are mounted in a house cannot be removed.

In other words, when an indoor concentration exceeds an environmental standard or guideline, some or other countermeasures such as change of causal furniture or changed of causal building materials by renovation is required, but it is highly demanded by a resident to take countermeasures capable of obtaining the optional performance cost.

In this case, it is important to recognize the extent of effect of specific chemical substance(s) released from individual emission sources on the indoor concentration.

For example, it can be said that the effect on the indoor concentration is small when an emission rate per unit area is small though a surface area is relatively large, whilst effect on the indoor concentration is small when the emission rate is small though the surface area is large. In practice, however, it was impossible to know the extent for the effect of the individual emission sources.

### DISCLOSURE OF THE INVENTION

### Subject to be Solved by the Invention

Therefore, a technical subject of the invention is, firstly, to quantify the effect of individual emission sources in indoor environment contaminated with hazardous chemical substances such as formaldehyde emitted from indoor emission source including furniture and building materials and provide fundamental data to simulate the change of indoor environment after the indoor emission source is removed or changed.

To solve the technical subject, the present invention provides a method of checking indoor environment that is adapted to output fundamental data for evaluating the effect of each indoor emission source that releases hazardous chemical substances on an indoor environment, characterized by calculating a emission rate of each emission source. chemical substances chemical substances

### EFFECT OF THE INVENTION

When the emission rate Fn of the chemical substances emitted from each indoor emission source is measured, since the emission rate Fn is a weight per unit area and unit time, the total emission amount: Qn=Fn×Sn per unit time of each emission source is determined by calculating the product thereof with the surface area Sn of each general source.

Then, based on the result, the indoor concentration Cn of the specific chemical substance can be calculated according to the following equation.

Cn(t)=(1−exp(−Nxt)/(Cout+(Qn/N/V))+C(t₀)×exp(−Nxt)

### N: ventilation rate

t: time

### Cout: outdoor concentration of specific chemical substance

### Qn: total emission amount from each indoor emission source

### V: room volume

The effect of the emission source on the indoor environment can be evaluated according to the emission rate of each emission source.

Accordingly, by comparing the individual indoor concentration with a predetermined indoor environmental guide line value and indicating the same in a multi-stage, for example, as 5-stage evaluation or 10-stage evaluation as described in claim 2, the extent of the effect of the indoor emission source, for example, whether it conforms the environmental standard or not in a conceptional manner.

Further, as described in claim 3, a contribution factor Kn represented by the total emission amount of each emission source in the total emission amount of indoor emission sources may be calculated instead of the individual indoor concentration by the following equation and the contribution factor Kn may be outputted as fundamental data:

Kn=Qn/ΣQ×100(%)

The contribution factor exhibits the ratio of the emission amount from each of the indoor emission sources for the total indoor concentration. Thus, the indoor concentration can be improved remarkably by reducing the emission from an emission source which has large contribution factor.

Further, as described in claim 4, the change of the indoor concentration with time in a closed room can be calculated for the current indoor environment. By simulating the indoor concentration after the removal of an emission source, as described in claim 5, We can judge the adequacy of the measurement. Particularly, the result of simulation can be clearly recognized at a glance when the current indoor concentration and the predicted indoor concentration curve are graphically indicated on an identical graph.

### BEST MODE FOR PRACTICING THE INVENTION

The present invention intends to calculate fundamental data for evaluating the effect of an indoor emission source that releases hazardous chemical substances to the indoor environment.

A recommended example for the practice of the present invention is described as followings.

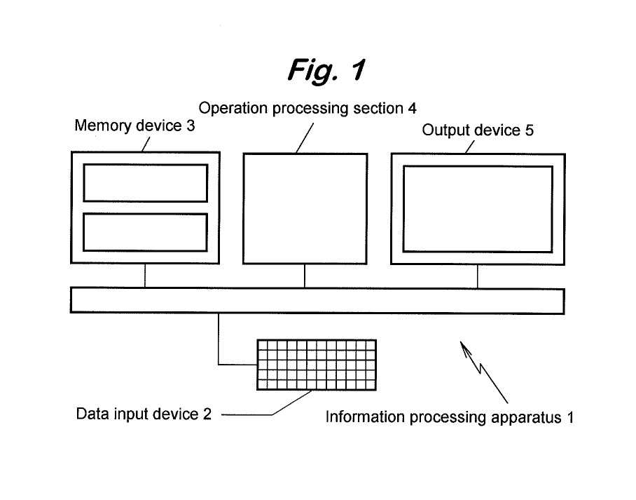

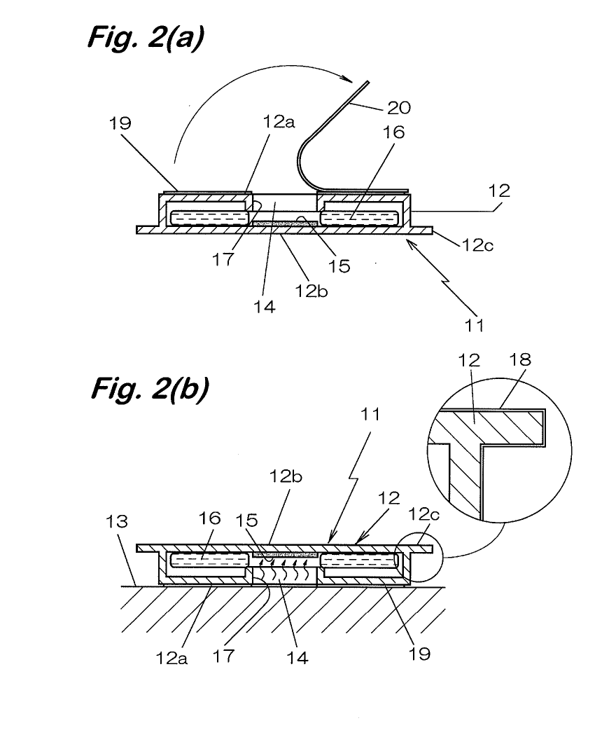

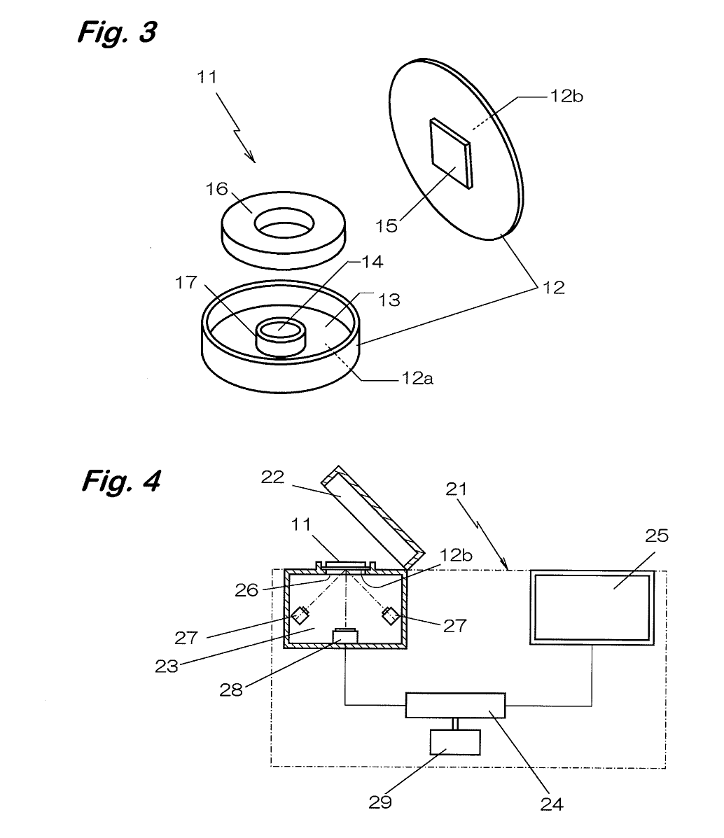

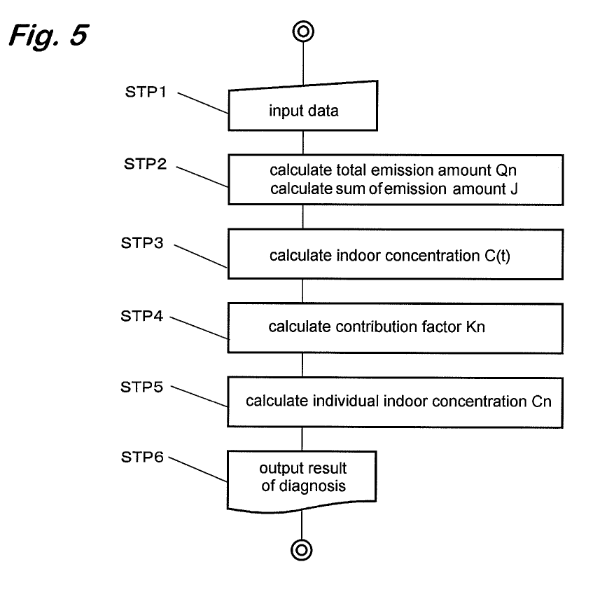

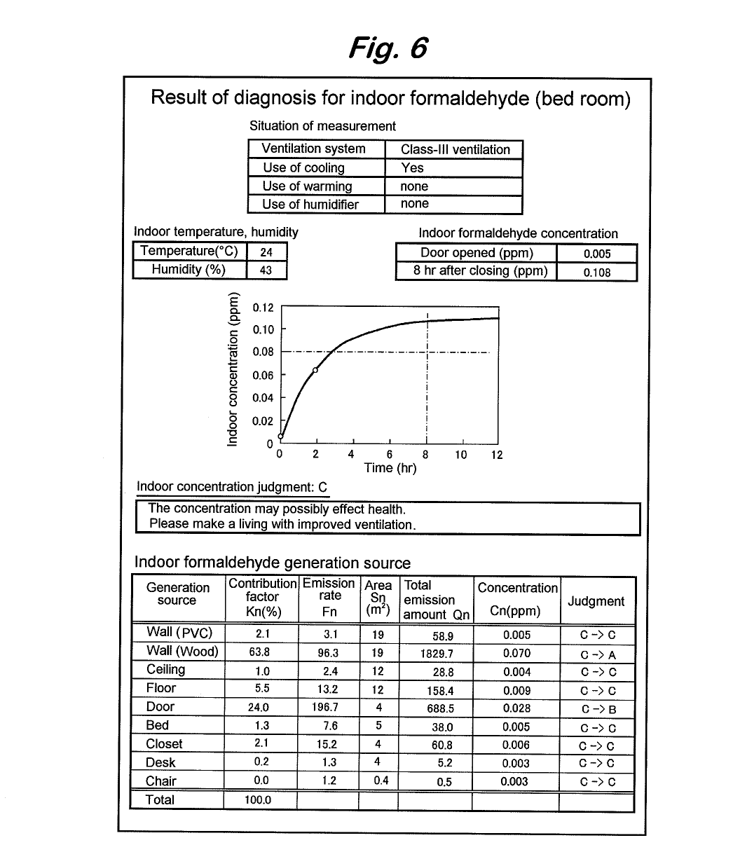

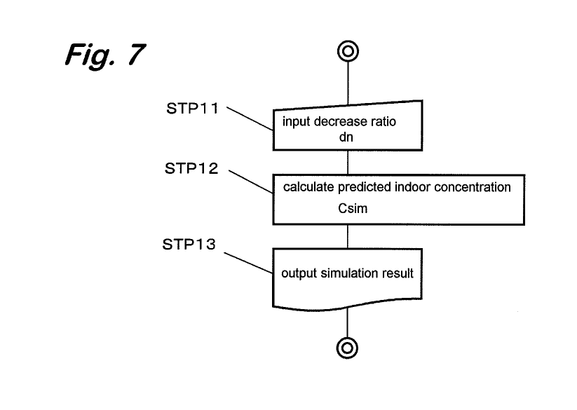

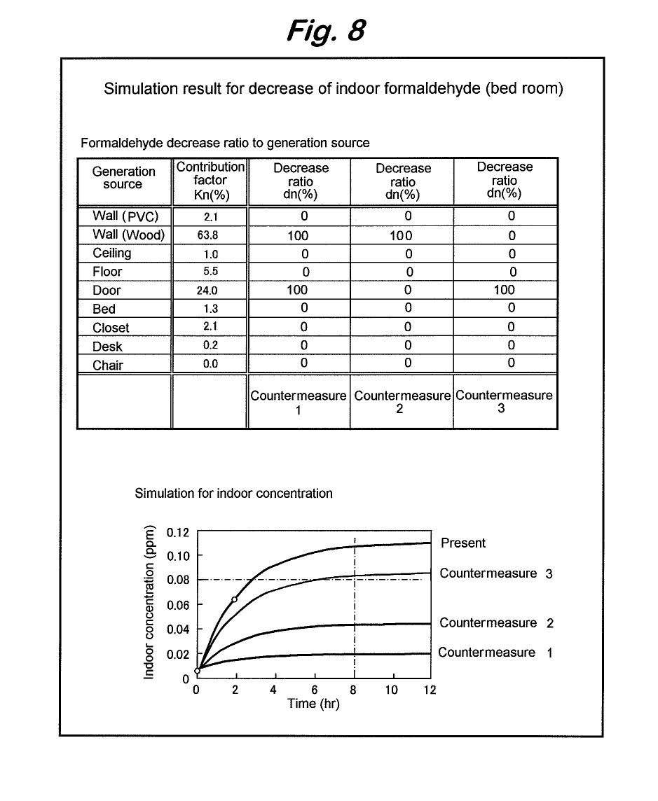

FIG. 1 shows an example of information processing tool used for an indoor environment diagnosis method according to the invention, FIG. 2 shows a cross sectional diagram of an example of a passive type emission flux sampler used in the invention, FIG. 3 shows an exploded diagram thereof, FIG. 4 is an explanatory diagram of an example of a device to measure the emission rate, FIG. 5 is a flow chart showing procedures of an indoor environment diagnosis method according to the invention, FIG. 6 is an explanatory view showing an example of a report showing the result of diagnosis, FIG. 7 is a flow chart showing procedures of simulation, and FIG. 8 is a graph showing the result of simulation.

By using this diagnosis method for the indoor environment, the effect of each indoor formaldehyde emission source can be estimated. For the calculation, the indoor concentration, the volume of the room, the surface area Sn of each indoor emission source and an emission rate Fn per unit area and unit time from each indoor emission source such as furniture, floor surfaces, wall surfaces, and ceiling surfaces, measures have to be obtained and entered to an information processing apparatus 1 such as a personal computer.

The information processing apparatus 1 includes a data input device 2 for inputting predetermined data, a memory device 3 for storing the data and a data processing program, etc., an operation processing section 4 for data processing in accordance with the program, and an output device 5 such as a display or a printer that outputs the result of processing.

When various necessary data are inputted to the information processing apparatus 1, the effect of an indoor emission source on the indoor environment is evaluated by a predetermined diagnosis program PRG1, and simulation for the change of the indoor concentration upon decreasing the emission amount of an indoor emission source is executed by a simulation program PRG2.

Before starting the processing, various necessary data are at first measured.

For each of the emission sources, for example, those portions where different building materials are used though they are present on an identical wall surface, are regarded as different emission sources and the emission rate Fn and the surface area Sn are measured respectively.

Further, the indoor concentration C(t) in a closed room is determined as a time function in which an indoor concentration C(t) in a state where windows are opened fully and an indoor concentration C(t₁) at the instance a predetermined time (30 min to 2 hours) has been elapsed in a state of closing the windows are measured and calculation is conducted based on the two data.

An emission rate Fn of formaldehyde emitted from each indoor emission source is measured, for example, by using a passive type emission flux sampler 11 shown in FIGS. 2 and 3.

In the passive type emission flux sampler 11, a hollow casing 12 having gas barrier property is formed into a hollow disk-like shape, a bottom surface 12a thereof is formed with an opening 14 for taking a chemical substance emitted from an inspection object 13 into the casing 12 in a state of bonding the bottom surface 12a to the inspection object 13, and a test piece 15 that conducts color change reaction with the chemical substance under a humid circumstance is bonded to the inner surface of the casing 12 being opposed to the opening 14.

Thus, a distance from the surface of the inspection object 13 to the test piece 15 can be kept constant in a state of bonding the flux sampler 11 to the inspection object 13.

Further, the hollow casing 12 is entirely formed transparent such that the color change of the test piece 15 can be observed from the outside in a state being bonded to the inspection object 13 as it is, and the side opposite to the bottom surface 12a constitutes an observation section 12b for observing the test piece 15 from the rear face, and a flange 12c is formed to the outer peripheral edge such that bonding and detachment can be conducted easily.

In the test piece 15, INT (p-iodo-nitrotetrazolium violet) as a chromophoric agent and two types of enzymes, i.e., dehydrogenase and diaphorase as a reaction catalyst are supported on a paper substrate sheet, for example, of about 1 cm×1 cm size.

Thus, when formaldehyde is in contact with the test piece 15 wetted with water, hydrogen of formaldehyde is dissociated by the dehydrogenase and they are decomposed into formic acid and NADH (nicotinamide adenine dinucleotide) and NADH and INT are reacted by diaphorase to decrease INT and develop a color.

In the casing 12, annular water retaining paper (water retaining member) 16 is disposed so as to surround a flow channel from the opening 14 to the test piece 15, which sucks a water droplet upon dripping the water droplet from the opening 14 into the casing 12 during measurement to keep the test piece 15 in a humid circumstance.

Further, in the opening 14, an annular rib 17 extends from the end edge thereof to the inside of the casing 12, by which the water droplet dripped from the opening 14 is guided with no stagnation by the surface tension of the water droplet to the water retaining paper 16 and it guides the chemical substance emitted from the inspection object 13 straight to the test piece 15 which is disposed being opposed to the opening 14 and causes the color change reaction more accurately in accordance with the emission rate thereof.

In this embodiment, the hollow casing 12 is made of a plastic material of about 0.5 mm thickness at a size of: diameter×thickness=about 2 cm×3 mm, with a diameter of the opening 14 being of about 5 mm.

In a case of using the plastic casing 12 of such a thickness, since formaldehyde permeates the plastic, a gas barrier film 18 such as a transparent DLC film (diamond like carbon film) or a vapor deposited silica film is vapor deposited to at least one of the outer surface or the inner surface of the casing 12 in order to enhance the gas barrier property against formaldehyde. A DLC film is formed in this embodiment.

Since the DLC film has an extremely high gas barrier property to formaldehyde, formaldehyde contained in indoor air does not permeate the casing 12 and change the color of the test piece 15 but only the released flux of formaldehyde released from the inspection object 13 can be measured accurately.

For the hollow casing 12, any material can be used such as glass or the like not being restricted to the plastic material and, in a case of using glass, since its gas barrier property is high by nature, it is not necessary to form a gas barrier film.

Then, an annular adhesive layer 19 is formed along the periphery of the opening 14 at the bottom surface 12a of the hollow casing 12, and a circular aluminum sheet 20 is bonded to the adhesive layer 19 to air tightly seal the opening 14 so that moisture does not intrude into the casing 12 in a preserved state.

In a case of measurement by using the flux sampler 11, as shown in FIG. 2(a), the aluminum sheet 20 is peeled with the opening 14 being upward, a water droplet is dripped from the opening 14 into the casing 12 to moisten the test piece 15, and the water retaining paper 16 is also wetted so as to maintain the test piece 15 in a humid circumstance during measurement.

In this case, since the annular rib 17 is formed to the opening 14, the water droplet flows smoothly into the casing 2 without staying at the end edge of the opening 14 by the surface tension of the water droplet.

Then, as shown in FIG. 2(b), the bottom surface 12a is bonded to an inspection object 13 such as a wall surface, floor surface, ceiling surface, or furniture.

In this case, even when it is bonded with the opening 14 being downward, since the water droplet in the casing 12 is dammed by the annular rib 17 formed to the opening 14, it does not flow out from the opening 14.

In this state, the chemical substance emitted from the inspection object 13 passes through the opening 14 and is taken into the casing 12, guided along the flow channel formed with the annular rib 17 and reaches the test piece 15 disposed in front thereof.

Then, after lapse of a predetermined time (30 min to 2 hours), the test piece 15 turns deep red in a place where the emission flux is large and turns pale red in a place where it is small, and scarcely changes color in a place where it is nearly equal to 0.

Accordingly, the emission flux can be measured in accordance with the color of the test piece 15 in the same manner as described above.

In this case, it is possible to read the emission rate by a previously prepared color chart but, for making it more accurate, the color change of the specimen 15 may be optically read by an emission rate measuring device 21 shown in FIG. 4.

FIG. 4 shows a measuring apparatus of emission flux to calculate the emission flux according to the invention.

The measuring apparatus 21 of this embodiment is adapted to measure the emission flux by using the flux sampler 11 described previously, in which a light shielding chamber 23 is formed to the inside of a light shielding cap 22 for optically measuring the color change of the test piece 15 and includes an operation processing device 24 for calculating the emission flux based on the detected color change and a liquid crystal display 25 for displaying the value thereof.

In the light shielding chamber 23, there are disposed a setting stage 26 for positioning the flux sampler 11, a light source 27 for irradiating a measuring light to the observing section 12b of the flux sampler 11, and an optical sensor 28 for detecting the intensity of reflection light from the observation section 12b of the flux sampler 11.

When the flux sampler 11 is set to the setting stage 26 with the observation section 12b being downward, a measuring light is irradiated from the light source 27 disposed below the setting stage 26 to the position for the test piece 15.

Since the test piece 15 reacts with formaldehyde to discolor red to red purple, the light source 27 uses an LED that outputs, as a measuring light, a green light in a complementary color relation therewith, and the center wavelength of the measuring light is selected to 555 nm in this embodiment.

A photodiode having peak sensitivity at a wavelength of 500 to 600 nm is used as the optical sensor 28. In a case where the amount of emission flux of formaldehyde is large, since the test piece 15 turns to deep color to absorb the measuring light, the intensity of the reflection light detected by the optical sensor 28 is lowered. On the other hand, in a case where the emission flux is small, since the test piece 15 is less discolored and absorbs less measuring light, the intensity of the reflection light increases relatively.

The operation processing device 24 calculates the absorption along with color change based on the intensity of the reflection light to calculate the emission rate based on the absorption.

At first, the absorption P is calculated according to the following equation:

P [1−L₁/L₀]×100(%)

L₀: intensity of reflection light of the test piece 15 before reaction or standard white color.

L₁: intensity of reflection light of the test piece 15 after reaction.

Then, a relation between the emission rate Fn and the absorption Pn is stored in an absorption-emission rate translation table 29 based on the absorption Pn of the sampler 11 measured by a known standard emission rate Fn, and the emission rate Fn is determined with reference to the absorption-emission rate translation table 29 based on the absorption P calculated for the flux sampler 11 after the reaction.

The absorption-emission rate translation table 29 may be represented by a function: Fn=f(Pn) or may store the translated values thereof in the form of a numerical table.

With such a constitution, since the emission rate Fn can be outputted as a numerical value, the emission rate can be calculated accurately for a subtle color change of the test piece 15 even in a case where comparison with the color chart is difficult.

As described above, after measuring the emission rate Fn per unit area and unit time of a specific chemical substance released from each of indoor emission sources such as furniture, floor surfaces, wall surfaces, and ceiling surfaces of a room in which the indoor concentration is high, and measuring the surface area Sn based on the size for each of the emission sources, such values are inputted into the information processing apparatus 1 such as a personal computer.

FIG. 5 is a floor chart showing processing procedures of a diagnosis program PRG1. At step STP1, indoor concentrations C(t₀) and C(t₁) for formaldehyde actually measured in a room as an object of measurement, a emission rate Fn from each of indoor emission sources such as furniture and building materials of the room, and the surface area Sn thereof are inputted.

Upon completion of the input, it goes to step STP2, the emission amount Qn from each indoor emission source is calculated according to:

Qn=Fn×Sn

which is stored in a predetermined memory region, the sum of the emission amounts is calculated according to:

J=ΣQn

which is stored in a predetermined memory region.

Then, it goes to step STP3, and a time function C (t) of the indoor concentration is calculated based on the indoor concentrations C(t₀) and C(t₁) of formaldehyde according to the following equation:

C(t)=(1−exp(−Nxt)/(Cout−(J/N/V))+C(t₀)×exp(−Nxt)

N: ventilation rate

t: time

Cout: outdoor formaldehyde concentration

J: sum of emission amount

V: room volume

Since Cout can be measured and only N is unknown, the value for N can be determined by taking time t on the abscissa and the indoor concentration C(t) on the ordinate, and fitting N so as to pass two points:

(t,C(t))=(t₀,C(t₀))

(t,C(t))=(t₁,C(t₁))

Further, at step STP4, a contribution factor Kn represent by the emission amount from each emission source for the sum of emission amount of formaldehyde from the indoor emission sources is calculated according to:

Kn=Qn/J×100(%)

and this is stored in a predetermined memory region.

Then, at step STP5, assuming that each of the indoor emission sources is placed individually in the room the individual indoor concentration Cn of a specific chemical substance is determined based on the equation for the indoor concentration C(t) determined at step STP3 by using the emission amount Qn of the emission source instead of the total for the emission amount as:

Cn(t)=(1−exp(−Nxt)/(Cout+(Qn/N/V))+C(t₀)×exp(−Nxt)

N: ventilation rate

t: time

Cout: outdoor formaldehyde concentration

Qn: total emission amount from each indoor emission source

V: room volume

Since the indoor concentration is based on the concentration at the instance after lapse of 8 hours in a state of closing the room, a value Cn(480) is used by substitution of: t=480 (min).

Since the individual indoor concentration Cn is a value for the indoor concentration of a specific chemical substance attributable to each indoor emission source, the effect of the emission source on the indoor environment can be evaluated by the value

Accordingly, at step STP6, by evaluating the value at six ranks from AAA to D as described below by comparing the value, for example, with an indoor environment guide line value defined by an academic society, the result can be checked simply even with no sufficient knowledge for information regarding the emission amount of formaldehyde.

AAA: 0.008 ppm or less

AA: 0.008 to 0.004 ppm

A: 0.04 to 0.08 ppm

B: 0.08 to 0.10 ppm

C: 0.10 to 0.16 ppm

D: 0.16 ppm or more

In this case, a notation system of showing from the current evaluation score to an evaluation score which will be reached when the indoor emission source is removed is used for easier understanding. For example, in a case where the current evaluation score is C for an article of furniture and it is reduced to A when the same is removed, it is described as in the form of “C->A”.

Evaluation upon removing the furniture in this case may be conducted by calculating the indoor concentration Csim on every indoor emission source for the decrease ratio of 100% in accordance with a simulation program PRG2 to be described later and evaluating the concentration at the six ranks from AAA to D as described above.

At step STP7, the result of diagnosis is outputted to complete the processing.

FIG. 6 shows an example of the result of diagnosis, which shows a case of measurement on a bed room in which walls (with vinyl chloride wall paper), walls (boarded), ceilings, floors, doors, beds, closets, and chairs constitute indoor emission sources.

As the result of analysis, the indoor concentration C(t) calculated at step STP3 is graphically indicated and indicates the indoor concentration of formaldehyde after lapse of a predetermined time in a closed state is shown.

Further, it outputs contribution factor Kn, emission rate Fn, area Sn, emission amount Qn, and result of judgment in a table form on each of the emission sources.

According to the result of diagnosis, it can be seen from the graph that the concentration exceeds indoor environment guide line value (0.08 ppm) at the lapse of 4 hours after closing windows and reaches 0.08 ppm at elapse of 8 hours as the criterion for the judgment.

Then, for executing simulation program PRG2 to conduct simulation of the indoor concentration in a case of applying a countermeasure to the indoor emission source, a decrease ratio dn is set to each of the indoor emission sources at first.

The decrease ratio dn can be set as five ranks from 0 to 100% such that it is 0% in a case where no countermeasure is applied and as 25%, 50%, 75% and 100% in a case where decrease can be expected by exchange or discarding.

It can be seen that the contribution factor Kn is as high as 63.8% for the wall (boarded) and 24% for the door and formaldehyde at about 90% for the total is emitted from the two emission sources.

Then, simulation is conducted in a case of replacing both of them with those of non-formaldehyde specification and in a case of replacing them individually with non-formaldehyde specification.

In this case, the decrease ratio in a case of replacing with the non-formaldehyde specification is assumed as 100% and the decrease ratio in a not replacing case is assumed as 0%.

FIG. 7 shows processing procedures of the simulation program PRG2. When the decrease ratio dn is inputted on each emission source at step STP11, a predicted indoor concentration Csim(t) is calculated at a step STP12.

Also, the decreased emission amount: Jsim=ΣQn−dn is used instead of the total emission amounts based on the equation calculated according to the step STP3:

Csim(t)=(1−exp(−Nxt)/(Cout−(Jsim/N/V))+C(t₀)×exp(−Nxt)

N: ventilation frequency

t: time

Cout: outdoor formaldehyde concentration

Jsim: decreased sum of emission amount

V: room volume

Then, at step STP13, the result is superimposed on a graph plane identical with that for the indoor concentration curve and indicated graphically in another color to complete the processing.

Thus, It can be observed at a glance how the indoor concentration has been decreased and it can be simulated easily as to whether the concentration can be lowered to the indoor environmental guide line value or less even or not in a state of closing windows.

FIG. 8 shows the result of the simulation and it can be seen that the predicted indoor concentration curve Csim(t) can be decreased further more from the current indoor concentration C(t).

In a case of replacing only the wall (boarded) with a non-formaldehyde specification, the indoor concentration is 0.04 ppm which is decreased to lower than the indoor environment guide line value of 0.08 ppm even in a case of closing windows for 8 hours or more and the evaluation A can be attained by the procedure alone.

Further, it can be seen that in a case of replacing only the door with the non-formaldehyde specification, the indoor concentration is 0.085 ppm which slightly exceeds the indoor environmental guide line and corresponds to the evaluation C. However, in a case of replacing both the wall (boarded) and the door with the non-formaldehyde specification, the indoor concentration of formaldehyde is lowered to about 0.02 to attain the evaluation AA.

### INDUSTRIAL APPLICABILITY

As has been described above, the present invention is applicable to the use of diagnosing the indoor environment by outputting the fundamental data for evaluating the effect of each indoor emission source that emits a hazardous chemical substance to the indoor environment.

### BRIEF DESCRIPTION OF THE DRAWINGS

[FIG. 1] is an explanatory view showing an example of information processing means used for the method of the invention.

[FIG. 2] is a cross sectional view showing an example of an emission flux sampler used for the method of the invention.

[FIG. 3] is an exploded view thereof.

[FIG. 4] is an explanatory view showing an example of a emission rate measuring apparatus.

[FIG. 5] is a flow chart showing processing procedures of the method of the invention.

[FIG. 6] is an explanatory view showing an example of a report showing the result of diagnosis.

[FIG. 7] is a flow chart showing processing procedures of simulation.

[FIG. 8] is an explanatory view showing an example of a report showing the result of simulation.

### DESCRIPTION OF REFERENCES

1 information processing apparatus
2 data input device
3 memory device
4 operation processing section
5 output device

1. A method of checking an indoor environment of calculating fundamental data for evaluating the effect of each indoor emission source that releases a hazardous specific chemical substance on the indoor environment, characterized by:
calculating a emission amount of each emission source per unit time based on a emission rate of a specific chemical substance released from each emission source per unit area and unit time and a surface area for each of emission sources; and calculating, based on the result an individual indoor concentration of the specific chemical substance as fundamental data when it is assumed that only each emission source is individually placed indoors as the fundamental data.

2. A method of checking the indoor environment according to claim 1, wherein the individual indoor concentration is evaluated by multi-stage in comparison with a predetermined guideline value for the indoor environment.

3. A method of checking an indoor environment of calculating fundamental data for evaluating the effect of each indoor emission source that emits a hazardous specific chemical substance on the indoor environment, characterized by:
calculating a emission amount of each emission source per unit time based on a emission rate of a specific chemical substance emitted from each emission source per unit area and unit time and a surface area of each of emission sources; and calculating a contribution rate represented by a emission amount of each emission source for the total emission amount of the specific chemical substance from emission sources as the fundamental data.

4. A method of checking the indoor environment according to claim 1, wherein an indoor concentration curve representing the change of indoor concentration with time is calculated in a state of closing a room based on an indoor concentration of the specific chemical substance measured in the room as an object of measurement.

5. A method of checking the indoor environment according to claim 4, wherein a predicted indoor concentration curve is calculated when a emission amount of any indoor emission source is decreased.

6. A method of checking the indoor environment according to claim 5, wherein the indoor concentration curve and the predicted indoor concentration curve are graphically shown on one identical graph plane.

7. A method of checking the indoor environment according to claim 3, wherein an indoor concentration curve representing the change of indoor concentration with time is calculated in a state of closing a room based on an indoor concentration of the specific chemical substance measured in the room as an object of measurement.

8. A method of checking the indoor environment according to claim 7, wherein a predicted indoor concentration curve is calculated when a emission amount of any indoor emission source is decreased.

9. A method of checking the indoor environment according to claim 8, wherein the indoor concentration curve and the predicted indoor concentration curve are graphically shown on one identical graph plane.
